SEQUENCE LISTING (5 sequences)

SEQ ID NO: 1 - DNA, 1371 bp, Homo sapiens
  misc_feature: Incyte ID No 238469.9
cttactccactttgcagtgagttctagtataacttctattccagtgagatcaaatggttaaggactccagaaaagaaatttggtgaacacaaagggtttgtcatactttaacttttgtccctttcccaggtggcccatgcttctaggccagcctcccaattcctagcttgcttcatgctcctggcctttgcctactcaaaacaatctgtcctccccaccctgtcatctctgttcagagtaaatacctccctccctctgcttttgccctaagttattttttcaactcacctaacatctaacttggctttgcagctctgtttctccaaaaccctctctgcctgtttcctcccaagctatctatgaccagtgccagaaaccagaaaatcttcttggaagttgcactctttgcttctcttccctctcctttatccttccacttggaatctctctccttctacctgtctctatctgctgcaatcctgttcatctttccaggtacattcctcactttttcatctttaagaagtcctctcagaaactgcataggattttttcatgatatatgccatattccactgtgtaatgtagattacaccacagggtttttaatttcctaaaggcaagagctataacctagtcatcttcctctcctaagtggggagtatcaccatgatttgaccaagtagctatgatatgggtgctatctccataaatgaatgagcagtgaggaaaaaggagatgattatgagtgaatgaagaatcttaataaggaaagtttattccacagtgaagcagtgttggtggctccaaaacctctctttaggaaggatttaggacagcatcctaatcaaaagggcctggaagcactttataaaagagagagacaagatcgcatgtcaaactagaaggaaggaggtgagaggagatagggctccagagtggagcaagccccttctgtccccttgaacttcctgccggtgcatgggttacctctcattaaatttaatagtacttgttgctttggtgtagtgaaatgaatgccttgatgaaattgcattgcaccatttttgaaagagagaatactcaaacgtgtcacttctgtttcttgcaagcaactgtgatcctgagctgtgcacacttctggttgggattatttctggtttctacttcctgtttgaagatgtggcatggagagtgctctgctttgacctgaagtattttatctatcctcagtctcaggacactgttgatggaattaaggccaagcacatctgcaaaaaagacattgctggaggaggtgcaaagagctggaaaccaagtctccagtcctgggaaaagcagtggtatggaaaagcaatggaaagagcattt

SEQ ID NO: 2 - DNA, 1833 bp, Homo sapiens
  misc_feature: Incyte ID No 348845.2ext (=348845.3 IBOB rel5) (1833nt)
tttccttgaaattaagttcaggtttgtctttgtgtgtaccaattaatgacaagaggttagatngcagtaatgctagatggcaaagagaaagtatgttttgtgtcttcaattttgctaaaaataacccagaacatggataattcatttattaattgattttggtaagccaagtcctatttggagaaaattaatagtttttctaaaaaagaattttctcaatatcacctggcttgataacatttttctccttcgagttcctttttctggagtttaacaaacttgttctttacaaatagattatattgactaccactcactgatgttatgatattagtttctattgcttactttgtatttctaattttaggattcacaatttagctggagaactattttttaacctgttgcacctaaacatgattgagctagaagacagttttaccatatgcatgcattttctctgagttatattttaaaatctatacatttctcctaaatatgggaggaaatcactggcatcaaatgccagtctcagacggaagacctaaagcccatttctggcctggagctacttggctttgtgacctatggtgaggcataagtgctctgagtttgtgttgcctcttttgtaaaatgagggtttgacttaatcagtgattttcatagcttaaaatttttttgaagaacagaactttttttaaaaacagttagatgcaaccatattatataaaacagaacagatacaagtagagctaacttgctaaagaaaggatggaggctctgaagctgtgacttcattatcccttaatactgctatgtcctctgtagtaccttagatttctatgggacatcgtttaaaaactattgtttatgcgagagccttgctaatttcctaaaaattgtggatacattttttctcccatgtataattttctcaccttctatttaaaaagaaaaaaaaagtcagtgtagtatttacatattttaccctataaggagctaacataacttttgatttagtgttattcataaaattaggttagcagtttattaaccttttgtatttgctctggcaatgtttaatatctcataagctatacacacctcgaagccatcaatgacaaccttttcttgctgaatagaacagtgattgatgtcatgaagacaattttatctccttttgccttccataatttgtaccaggttatataatagtataacactgccaaggagcggattatctcatcttcatcctgtaattccagtgtttgtcacgtggttgttgaataaatgaataaagaatgagaaaaccagaagctctgatacataatcataatgataattatttcaatgcacaactacgggtggtgctgaactagaatctatattttctgaaactggctcctctaggatctactctatgattttaatctttatagtatgaagttagtaatagcatcagaaaaaaaaggtaaacaaattgctcctgtggagatgattgggcatcacatggtggtttgagctgatacacccaacacttgagctcactggcaacagtaccagattttcaccgctatgcctcctttcactctgggagtctttccagaggtcttgcactcgggagagcatgctcaggtttccccagctctacagaatcacccagaatgccaaagacttcaacacaaggatactgtgttgaccagtggtcatgccactgcctgttgatttgttgaaaatattgtttacacgtatgttcttgttactggattgtcagaaagctgggttttggagactgcagcttggactaaattcagtcatctggctgtctggggg

SEQ ID NO: 3 - DNA, 2043 bp, Homo sapiens
  misc_feature: Incyte ID No 411152.2ext =IBOB rel5 (2043nt)
gtatacattctttattaatcattttgcttccaaccccatttagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctacacatcaaaaagaaatagataaaataaatggaaaattagaagagtctcctgataatgatggttttctgaaggctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaaggttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaaccagagctctgcagtgtgaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgcaggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcatcaccttatgttgaaaatcttaccaatagtctgtgtcaacagaatacttattttagaagaaaaattcatgatttcttcctgaagcctacagacataaaataacagtgtgaagaattacttgttcacgaattgcataaagctgcacaggattcccatctaccctgatgatgcagcagacatcattcaatccaa

SEQ ID NO: 4 - DNA, 651 bp, Homo sapiens
  misc_feature: Incyte ID No 1135407.1ext (=IBOB rel 5 template 242151.1)(651nt)
cacttttgatcttcccttcagcgaccttgaagcctttgatatatcccccttttaactctaccaccttcatccctctgttcattctgccaggcttttcatttcccacttcaacctctcagctcccaacagtcacttgaactatagaccccttggcctccattgggggctctccttaagagactgagggatccctgagggatccccaaaagcaatgattagaggctgaaaacagaaaaaaaactttggaaacagactggatgttttgtacactcagagaatccgacaacagctgctccagctgacacgtatccagctactggtcctgctgatgatgaagcccctgatgctgaaaccactgctgctgcaaccactgcgaccactgctgctcctaccactgcaaccaccgctgcttctaccactgctcgtaaagacattccagttttacccaaatgggttggggatctcccgaatggtagagtgtgtccctgagatggaatcagcttgagtcttctgcaattggtcacaactattcatgcttcctgtgatttcatccaactacttaccttgcctacgatatcccctttatctctaatcagtttattttctttcaaataaaaaataactatgagcaaaaaaaaaagaaaaaaaagg

SEQ ID NO: 5 - protein, 241 aa, Homo sapiens
  misc_feature: Incyte ID No 411152.2+2
MQKSVPNKALELKNEQTLRADEILPSESKQKDYEESSWDSESLCETVSQKDVCLPKATHQKEIDKINGKLEGSPVKDGLLKANCGMKVSIPTKALELMDMQTFKAEPPEKPSAFEPAIEMQKSVPNKALELKNEQTLRADQMFPSESKQKKVEENSWDSESLRETVSQKDVCVPKATHQKEMDKISGKLEDSTSLSKILDTVHSCERARELQKDHCEQRTGKMEQMKKKFCVLKKKLSEAK

(19) United States
(12) Patent Application Publication
(10) Pub. No.: US 2003/0104418 A1
(43) Pub. Date: Jun. 5, 2003

(54) Diagnostic markers for breast cancer

(76) Inventors: Chao Zhang, Moraga, CA (US); Behzad Mahini, Saratoga, CA (US); Michael G. Walker, Sunnyvale, CA (US)

Correspondence Address: INCYTE GENOMICS, INC., 3160 PORTER DRIVE, PALO ALTO, CA, 94304, US

(21) Appl. No.: 10/133,757

(22) Filed: Apr. 25, 2002

Related U.S. Application Data
(60) Provisional application No. 60/287,153, filed on Apr. 27, 2001.

(51) Int. Cl. C12Q 1/68; C07H 21/04; C12P 19/34

(52) U.S. Cl. 435/6; 435/91.2; 536/23.2

(57) ABSTRACT

The invention provides polynucleotides that are differentially expressed in breast cancer. The invention also provides a combination of polynucleotides, proteins encoded by the polynucleotides, and antibodies which specifically bind a protein, compositions, probes, expression vectors, and host cells. The invention also provides methods for the diagnosis, prognosis, treatment and evaluation of therapies for breast cancer.

This application claims benefit of provisional application Serial No. 60/287,153, filed Apr. 27, 2001.

### FIELD OF THE INVENTION

The invention relates to isolated polynucleotides and proteins that are highly expressed in breast tissue and co-expressed with known breast cancer diagnostic marker genes and proteins and useful for diagnosis, prognosis, treatment and evaluation of therapies for breast cancer.

### BACKGROUND OF THE INVENTION

Breast cancer is the most common cancer affecting women, and there are more than 180,000 new cases of breast cancer diagnosed each year. The mortality rate for breast cancer approaches 10% of all deaths in females between the ages of 45 and 54 (Gish (1999) AWIS Magazine 28:7-10). Survival rate varies from 97% for localized breast cancer with early diagnosis to 22% for advanced stage, metastatic disease. Classically, breast cancers have been categorized by histologic appearance and location of the lesion. The common categories include adenocarcinoma, ductal carcinoma, lobular carcinoma, in situ carcinoma, and infiltrating or invasive carcinoma, and each may involve inflammatory complications.

Although breast cancer may develop anytime after puberty, it is most common in postmenopausal women and relatively rare in men. The causes and genetic and environmental components of this disease are for the most part unknown, however, many breast cancers are sensitive to steroids, and estrogen or androgen may potentiate their growth.

Familial breast cancer accounts for 5% to 9% of known cases and is caused by mutations in two genes, BRCA1 and BRCA2. These diagnostic marker genes not only predispose a subject to breast cancer but may also be passed to offspring (Gish, supra). The vast majority of breast cancers are adenocarcinomas caused by noninherited mutations in breast epithelial cells. The expression of specific genes associated with breast cancer, for example, the relationship between expression of epidermal growth factor (EGF) and its receptor, EGFR (a member of the erbB family of proteins) to human mammary carcinoma has been well studied. Overexpression of EGFR, particularly coupled with down-regulation of the estrogen receptor, is a marker of poor prognosis. In addition, EGFR expression in breast tumor metastases is frequently elevated relative to the primary tumor, which suggests EGFR is involved in tumor progression and metastasis. This is supported by accumulating evidence that EGF affects metastatic potential through cell division and motility, chemotaxis, secretion, and differentiation.

Changes in expression of other members of the erbB receptor family have also been implicated in breast cancer. The abundance of erbB receptors, such as HER-2/neu, HER-3, and HER-4, and their ligands in breast cancer suggests their functional importance in the pathogenesis of the disease and their potential as targets for therapy (Bacus et al. (1994) Am J Clin Pathol 102:S13-S24). Other known breast cancer diagnostic markers include matrix G1a protein which is overexpressed is human breast carcinoma cells (Chen et al. (1990) Oncogene 5:1391-1395); maspin, a tumor suppressor gene down-regulated in invasive breast carcinomas (Sager et al. (1996) Curr Top Microbiol Immunol 213:51-64); CaN19, a member of the S100 protein family, all of which are down-regulated in mammary carcinoma cells; Zn-alpha 2-glycoprotein (Zn-α2) messenger RNA which is up-regulated by glucocorticoids and androgens in a specific set of human breast carcinomas (Lopez-Boado et al. (1994) Breast Cancer Res Treat 29:247-58); human mammoglobin (hMAM), a superior marker of breast cancer cells in peripheral blood (Grunewald et al. (2000) Lab Invest 80:1071-7); and bullous pemphigoid antigen (BPAG1), also known as “hemidesmosomal plaque protein”, which is not expressed in invasive breast cancer cells including carcinoma in situ (Bergstraesser et al. (1995) Am J Pathol 147:1823-39).

Cell lines derived from human mammary epithelial cells at various stages of breast cancer provide useful models to study the process of malignant transformation, cell division, and tumor progression. These cell lines have been shown to retain many phenotypic and molecular characteristics of the parental tumor for lengthy culture periods (Wistuba et al. (1998) Clin Cancer Res 4:2931-2938).

In that clinical procedures for breast examination are lacking in sensitivity and specificity, efforts are underway to develop gene expression profiles that may be used with conventional methods to improve diagnosis and prognosis (Perou CM et al. (2000) Nature 406:747-752). The present invention satisfies a need in the art by providing a plurality of expressed polynucleotides, their encoded proteins, and antibodies which specifically bind the proteins which may be used for the diagnosis, prognosis, treatment and evaluation of therapies for breast cancer.

### SUMMARY OF THE INVENTION

The invention provides a combination comprising a plurality of polynucleotides having the nucleic acid sequences of SEQ ID NOs: 1-4 that are differentially expressed in breast cancer and the complements of SEQ ID NOs: 1-4. In one embodiment, the combination is placed on a substrate. The invention also provides a method of using a combination to screen a plurality of molecules to identify at least one ligand which specifically binds a polynucleotide of the combination, the method comprising combining the substrate containing the combination with molecules under conditions to allow specific binding; and detecting specific binding, thereby identifying a ligand which specifically binds a polynucleotide of the combination. In one embodiment, the molecules are selected from DNA molecules, mimetics, peptides, peptide nucleic acids, proteins, RNA molecules, ribozymes, and transcription factors. The invention further provides a method for using a combination to detect gene expression in a sample containing nucleic acids, the method comprising hybridizing the substrate containing the combination to the nucleic acids under conditions for formation of one or more hybridization complexes; and detecting hybridization complex formation, wherein complex formation indicates gene expression in the sample. In one embodiment, the sample is from breast. In another embodiment, complex formation when compared to standards is diagnostic of a breast cancer selected from adenocarcinoma; ductal carcinoma; invasive, infiltrating, or metastatic (mets) carcinomas; lobular carcinoma; intraductal carcinoma; medullary, circumscribed, or in situ carcinoma; and an inflammatory complication of breast cancer.

The invention provides an isolated polynucleotide comprising a cDNA having a nucleic acid sequence selected from SEQ ID NOs: 1-4 and the complements thereof. In different aspects, each polynucleotides is used as probe, in an expression vector, and in assays for diagnosis, prognosis, and treatment of breast cancer. The invention further provides a composition comprising a polynucleotide and a labeling moiety. The invention still further provides a method for using a polynucleotide of the invention to screen a plurality of molecules to identify a ligand which specifically binds the polynucleotide, the method comprising combining the polynucleotide with a sample under conditions to allow specific binding;

recovering the bound polynucleotide; and separating the ligand from the bound polynucleotide, thereby obtaining purified ligand. In one embodiment, the molecules to be screened are selected from DNA molecules, mimetics, peptides, peptide nucleic acids, proteins, RNA molecules and transcription factors.

The invention provides a method for using a polynucleotide to detect gene expression in a sample containing nucleic acids, the method comprising hybridizing the polynucleotide to nucleic acids of a sample under conditions for formation of one or more hybridization complexes; and detecting hybridization complex formation, wherein complex formation indicates gene expression in the sample. In one embodiment, the polynucleotide is attached to a substrate. In another embodiment, gene expression when compared to standards is diagnostic of a breast cancer selected from adenocarcinoma; ductal carcinoma; invasive, infiltrating, or metastatic (mets) carcinomas; lobular carcinoma; intraductal carcinoma; medullary, circumscribed, or in situ carcinoma; and an inflammatory complication of breast cancer.

The invention provides a method for producing a peptide or protein. The invention provides a vector containing a polynucleotide having a nucleic acid sequence selected from SEQ ID NOs: 1-4, a host cell containing the vector, and using the host cell to produce a protein or peptide encoded by the polynucleotide, the method comprising culturing the host cell under conditions for expression of the protein; and recovering the protein so produced from cell culture.

The invention provides a purified protein comprising the amino acid sequence of SEQ ID NO: 5. The invention also provides a method for using a protein or peptide to screen a plurality of molecules to identify at least one ligand which specifically binds the protein. In one embodiment, the molecules to be screened are selected from agonists, antagonists, antibodies, DNA molecules, peptides, peptide nucleic acids, proteins including transcription factors, enhancers, and repressors, RNA molecules, and small drug molecules or compounds. The invention further provides a method of using a protein to purify a ligand.

The invention provides a method for using the protein to produce an antibody which specifically binds the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention provides purified antibodies which bind specifically to a protein. The invention also provides a method for using an antibody to detect expression of a protein in a sample, the method comprising combining the antibody with a sample under conditions for formation of antibody:protein complexes, and detecting complex formation, wherein complex formation indicates expression of the protein in the sample. In one aspect, the amount of complex formation when compared to standards is diagnostic of breast cancer.

The invention provides a method for immunopurification of a protein comprising attaching an antibody to a substrate, exposing the antibody to a sample containing protein under conditions to allow antibody:protein complexes to form, dissociating the protein from the complex, and collecting purified protein. The invention also provides an array upon which a polynucleotide encoding a protein, the protein, or an antibody which specifically binds the protein are immobilized. The invention also provides a composition comprising a polynucleotide, a protein, an antibody, or a ligand which has agonistic or antagonistic activity.

### BRIEF DESCRIPTION OF THE SEQUENCE LISTING AND FIGS.

The Sequence Listing provides SEQ ID NOs: 1-4, exemplary polynucleotides of the invention. Each sequence is identified by a sequence identification number (SEQ ID NO) and by the Incyte number with which the sequence was first identified.

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two polynucleotides, proteins, or antibodies on a substrate. At least one of the polynucleotides, proteins, or antibodies represents a control or standard, and the other polynucleotide, protein, or antibody of diagnostic or therapeutic interest. The arrangement of at least two and up to about 40,000 polynucleotides, proteins, or antibodies on the substrate assures that the size and signal intensity of each labeled complex, formed between each polynucleotide and at least one nucleic acid, each protein and at least one ligand or antibody, or each antibody and at least one protein to which the antibody specifically binds, is individually distinguishable.

A “combination” comprises at least two and up to about 8 sequences selected from the group consisting of SEQ ID NOs: 14 and their complements as presented in the Sequence Listing.

“Breast cancer” includes any tumor or neoplasia of the breast and specifically refers to adenocarcinoma; ductal carcinoma; invasive, infiltrating, or metastatic (mets) carcinomas; lobular carcinoma; intraductal carcinoma; medullary, circumscribed, or in situ carcinoma; and inflammatory complications of breast cancer.

“Differential expression” refers to an increased or up-regulated or a decreased or down-regulated expression as detected by absence, presence, or at least two-fold change in the amount of transcribed messenger RNA or translated protein in a sample.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses two-dimensional polyacrylamide electrophoresis (2D-PAGE, mass spectrophotometry (MS), enzyme-linked immunosorbent assays (ELISAs), radioimmunoassays (RIAs), and fluorescence activated cell sorting (FACS) or arrays and labeling moieties or antibodies to detect expression in a sample. The nucleic acids, proteins, or antibodies may be used in solution or attached to a substrate, and their detection is based on methods and labeling moieties well known in the art.

A “hybridization complex” is formed between a polynucleotide of the invention and a nucleic acid of a sample when the purines of one molecule hydrogen bond with the pyrimidines of the complementary molecule, e.g., 5′-A-G-T-C-3′ base pairs with its complete complement, 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-340). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Isolated or purified” refers to a polynucleotide or protein that is removed from its natural environment and that is separated from other components with which it is naturally present.

“Labeling moiety” refers to any reporter molecule whether a visible or radioactive label, stain or dye that can be attached to or incorporated into a polynucleotide or protein. Visible labels and dyes include but are not limited to anthocyanins, βglucuronidase, BIODIPY, Coomassie blue, Cy3 and Cy5, digoxigenin, FITC, green fluorescent protein, luciferase, spyro red, silver, and the like. Radioactive markers include radioactive forms of hydrogen, iodine, phosphorous, sulfur, and the like.

“Ligand” refers to any agent, molecule, or compound which will bind specifically to a complementary site on a cDNA molecule or polynucleotide, or to an epitope or a protein. Such ligands stabilize or modulate the activity of polynucleotides or proteins and may be composed of inorganic or organic substances including nucleic acids, proteins, carbohydrates, fats, and lipids.

“Markers for breast cancer” refers to polynucleotides, proteins, and antibodies which are useful in the diagnosis, prognosis, treatment or evaluation of therapies for breast cancer. This means that the marker is differentially expressed in samples from subjects predisposed to or manifesting breast cancer. The known breast cancer diagnostic marker genes used in co-expression analysis included Zn-alpha 2-glycoprotein (Zn-α2), human mammoglobin (hMAM), and bullous pemphigoid antigen (BPAG1).

“Polynucleotide” refers to an isolated cDNA. It may be of recombinant or synthetic origin, double-stranded or single-stranded, and combined with vitamins, minerals, carbohydrates, lipids, proteins, or other nucleic acids to perform a particular activity or form a useful composition.

“Probe” refers to a polynucleotide of the invention that hybridizes to at least one nucleic acid in a sample. Where targets are single stranded, probes are complementary single strands. Probes can be labeled for use in hybridization reactions including Southern, northern, in situ, dot blot, array, and like technologies or in screening assays.

“Protein” refers to a polypeptide or any portion thereof. An “oligopeptide” is an amino acid sequence from about five residues to about 15 residues that is used as part of a fusion protein to produce an antibody that specifically binds the protein.

“Sample” is used in its broadest sense as containing nucleic acids, proteins, antibodies, and the like. A sample may comprise a bodily fluid such as ascites, blood, lymph, saliva, semen, spinal, sputum, tears, and urine; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue or tissue biopsy; a tissue print; buccal cells, skin, a hair or its follicle; and the like.

“Specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which polynucleotides or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a polynucleotide or a protein encoded by the polynucleotide. Splice variants may be determined by BLAST score, wherein the score is at least 100, and most preferably at least 400. Allelic variants have a high percent identity to the polynucleotides and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid.

The Invention

The present invention identifies a plurality of polynucleotides that can serve as surrogate diagnostic markers for breast cancer. In particular, the method identifies polynucleotides cloned from mRNA transcripts which are differentially expressed in breast cancer and which co-express with known breast cancer diagnostic marker genes. These polynucleotides, the proteins or peptides which they encode, and antibodies which specifically bind the proteins are useful in diagnosis, prognosis, treatment, and evaluation of therapies for breast cancer.

The method disclosed below provides for the identification of polynucleotides that are expressed in a plurality of libraries. The polynucleotides originate from human cDNA libraries derived from a variety of sources. These polynucleotides can also be selected from a variety of sequence types including, but not limited to, expressed sequence tags (ESTs), assembled polynucleotides, full length coding regions, promoters, introns, enhancers, 5′ untranslated regions, and 3′ untranslated regions.

The cDNA libraries used in the analysis can be obtained from any human tissue including, but not limited to, adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, phagocytes, pituitary, placenta, pleura, prostate, salivary glands, seminal vesicles, skeleton, spleen, stomach, testis, thymus, tongue, ureter, and uterus.

The polynucleotides are highly specific to breast tissue and differentially expressed in association with breast cancers. The tissue distribution of 40,285 gene bins in 1222 libraries in the LIFESEQ GOLD database (release October 2000; Incyte Genomics, Palo Alto Calif.) were analyzed. The 40,285 gene bins represent genes that were detected in at least 5 of the 1292 libraries. The 1222 libraries include all surgical samples, biopsies, and cell line cDNA libraries and are the subset of 1292 libraries that had a unique tissue types. Those libraries which were constructed using tissues described as either mixed or pooled were not considered in this analysis.

In a preferred embodiment, the polynucleotides are assembled from related sequences, such as sequence fragments derived from a single transcript. Assembly of the polynucleotide can be performed using sequences of various types including, but not limited to, ESTs, extension of the EST, shotgun sequences from a cloned insert, or full length polynucleotides. In a most preferred embodiment, the polynucleotides are derived from human sequences that have been assembled using the algorithm disclosed in U.S. Pat. No. 9,276,534, filed Mar. 25, 1999, incorporated herein by reference.

Experimentally, differential expression of the polynucleotides can be evaluated by methods including, but not limited to, differential display by spatial immobilization or by gel electrophoresis, genome mismatch scanning, representational difference analysis, microarray analysis and transcript imaging. Any of these methods can be used alone or in combination to produce an expression profile; in the present case, the preferred method is presented below.

The Method

The method for identifying polynucleotides that exhibit a statistically significant expression pattern in breast, and specifically in breast cancer, is presented below. First, the presence or absence of a polynucleotide in a cDNA library is defined: a polynucleotide is present when at least one cDNA fragment corresponding to that polynucleotide is detected among the cDNAs of the library, and a polynucleotide is absent when no corresponding cDNA fragment is detected. This method was applied to the data in the LIFESEQ GOLD database (Incyte Genomics).

To determine whether a polynucleotide (G) is breast specific, two statistical tests are applied. In the first test, the significance of gene expression is evaluated using a probability method to measure a due-to-chance probability of expression. Two dichotomous variables are used to classify the 1222 cDNA libraries, X which determines whether G is present (P) or absent (A), and Y which determines whether the cDNA library is from breast (B) or not (θ). Occurrence data in the various categories is summarized in the following contingency table.

| | Breast | Non-breast |
|---|---|---|
| G present | PB | Pθ |
| G absent | AB | A |

If polynucleotide G is breast specific, a positive association between the two variables X and Y is expected; that is, a significant number of libraries should fall into the PB and Aθ categories. To evaluate the significance in statistical terms, the following question is asked: if the null hypothesis were true—that is, the presence of polynucleotide G were completely independent of whether the tissue is breast or not—how likely is it that the result occurred by chance. This is provided by applying the Fisher exact probability test and examining the P value (Agresti (1990) Categorical Data Analysis, John Wiley & Sons, New York N.Y.; Rice (1988) Mathematical Statistics and Data Analysis, Duxbury Press, Pacific Grove Calif.). The smaller the P value, the less likely that the association between X and Y is due-to-chance.

To illustrate, if a polynucleotide was detected in eight of the 1222 cDNA libraries and six of those were from breast, the corresponding contingency table would be:

| | Breast | Non-breast |
|---|---|---|
| G present | 6 | 2 |
| G absent | 40 | 1174 |

and the Fisher exact P value would be 5.4⁻⁰⁸, which indicates that the polynucleotide is breast specific.

In the second test, the EST counts of polynucleotide G from all libraries that were taken from the same tissue are combined, and the sum is used as a measure of the expression level in that tissue. In particular, the combined EST count of G in breast libraries (N_{GB}) is compared to the total number of ESTs for all polynucleotides which occur in breast libraries (NB) to derive an estimate of the relative abundance of G transcripts in breast. Similarly, the combined EST count of G in non-breast libraries (N_{GB}) is compared with the total number of ESTs in non-breast libraries (N_{GB}). These values are used to define a likelihood score

L=log2 (N_{GB}/N_{B})/(N_{Gθ}/N_{θ}),

which reflects how many times more likely it is for the transcript of polynucleotide G to be found in breast versus non-breast tissue. For the polynucleotide shown in the contingency table above, the respective counts are N_{GB}=11, N_{B}108756, N_{Gθ}=3, and N_{θ}=3556776, which give rise to L=log2(120)=6.91. Because the likelihood score is susceptible to the counting errors that exist in some libraries, the likelihood score is only used as a secondary measure.

In other words, polynucleotides with a significant Fisher exact P value of P<1e⁻⁵, are only considered to be breast-specific if L>5.5. This two-step filtering was found to select most polynucleotides known to function in breast without including any false positives. Note that the definition of L is flawed when N_{GB}=0 or N_{Gθ}=0. In this case, L>5.5 is considered only when N_{Gθ}and N_{GB}≠0.

Using this method to analyze 40,285 gene bins, those polynucleotides that exhibit significant association with breast cancer have been identified. Their expression patterns were compared with those of known breast cancer diagnostic marker genes using the Guilt-by-Association (GBA) analysis for co-expression patterns described by Walker et al. (1999; Genome Res 9:1198-203; incorporated herein by reference). The known breast cancer diagnostic marker genes highly significantly co-express with the polynucleotides of the invention. Therefore, the polynucleotides of the invention are useful as surrogate markers for the diagnosis, prognosis, treatment and evaluation of therapies for breast cancer, particularly adenocarcinoma; ductal carcinoma; invasive, infiltrating, or metastatic (mets) carcinomas; lobular carcinoma; intraductal carcinoma; medullary, circumscribed, or in situ carcinoma; and inflammatory complications of breast cancer. Further, a protein or peptide encoded by any of the polynucleotides can be used as a diagnostic, as a potential therapeutic, as a target for the identification or development of therapeutics, or for producing antibodies which specifically bind the protein or peptide. These antibodies are useful in the diagnosis, prognosis, and treatment of breast cancer.

Gene Expression Profiles

A gene expression profile comprises a plurality of polynucleotides and a plurality of detectable hybridization complexes, wherein each complex is formed by hybridization of one or more polynucleotides to one or more complementary nucleic acids in a sample. Assays for proteins and antibody arrays may also be used to produce an expression profile. The correspondence between mRNA and protein expression has been discussed by Zweiger (2001, Transducing the Genome. McGraw-Hill, San Francisco, Calif.) and Glavas et al. (2001; T cell activation up-regulates cyclic nucleotide phosphodiesterases 8A1 and 7A3, Proc Natl Acad Sci 98:6319-6342) among others.

In this invention, the polynucleotides are used as elements on a array to analyze gene expression. In one embodiment, the array is used to monitor the progression of disease. Researchers and clinicians can catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease; or treatment of the condition, disorder or disease. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

In one embodiment, the invention encompasses a combination comprising a plurality of polynucleotides having the nucleic acid sequences of SEQ ID NOs: 1-4 and the complements thereof. These polynucleotides have been shown by the methods of the present invention to have significant, specific, and differential expression in breast cancer. The invention also provides a polynucleotide and methods for using a polynucleotide selected from SEQ ID NOs: 1-4 and the complements thereof.

The polynucleotide or the encoded protein or peptide can be used to search against the GenBank primate (pri), rodent (rod), mammalian (mam), vertebrate (vrtp), and eukaryote (eukp) databases, SwissProt, BLOCKS (Bairoch et al. (1997) Nucleic Acids Res 25:217-221), PFAM, and other databases that contain previously identified and annotated motifs, sequences, and gene functions. Methods that search for primary sequence patterns with secondary structure gap penalties (Smith et al. (1992) Protein Engineering 5:35-51) as well as algorithms such as Basic Local Alignment Search Tool (BLAST; Altschul (1993) J Mol Evol 36:290-300; Altschul et al. (1990) J Mol Biol 215:403-410), BLOCKS (Henikoff and Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp 856-853).

Also encompassed by the invention are polynucleotides that are capable of hybridizing to SEQ ID NOs: 1-4. Conditions for hybridization (e.g., Ausubel, supra, unit 2 pp. 1-41 and unit 4, pp. 22-27) can be selected by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency, with buffers such as 5× SSC (saline sodium citrate) with 1% sodium dodecyl sulfate (SDS) at 60° C., which permits complex formation between two nucleic acid sequences that contain some mismatches. Subsequent washes are performed at higher stringency with buffers such as 0.2× SSC with 0.1% SDS at either 45° C. (medium stringency) or 68° C. (high stringency), to maintain hybridization of only those complexes that contain completely complementary sequences. Background signals can be reduced by the use of detergents such as SDS, sarcosyl, or TRITON X-100 (Sigma-Aldrich, St. Louis Mo.), and/or a blocking agent, such as salmon sperm DNA. Hybridization methods are described in detail in Ausubel (supra, units 2.8-2.11, 3.18-3.19 and 4-6-4.9) and Sambrook et al. (1989; Molecular Cloning A Laboratory Manual, Cold Spring Harbor Press, Plainview N.Y.)

A polynucleotide can be extended utilizing a partial nucleotide sequence and employing various methods such as PCR and shotgun cloning which are well known in the art. These methods can be used to extend upstream or downstream to obtain a full length sequence or to recover useful untranslated regions (UTRs), such as promoters and other regulatory elements. For PCR extensions, an XL-PCR kit (Applied Biosystems (ABI), Foster City Calif.), nested primers, and commercially available cDNA libraries (Invitrogen, Carlsbad Calif.) or genomic libraries (Clontech, Palo Alto Calif.) can be used to extend the sequence. For all PCR-based methods, primers can be designed using commercially available software (LASERGENE software, DNASTAR, Madison Wis.) to be about 15 to 30 nucleotides in length, to have a GC content of about 50%, and to form a hybridization complex at temperatures of about 68C to 72C.

In another aspect of the invention, the polynucleotide can be cloned into a recombinant vector that directs the expression of the protein, peptide, or structural or functional portions thereof, in host cells. Due to the inherent degeneracy of the genetic code, other DNA sequences which encode the same or a functionally equivalent amino acid sequence can be produced and used to express the protein encoded by the polynucleotide. The nucleotide sequences of the present invention can be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides can be used to engineer the nucleotide sequences. For example, oligonucleotide-mediated site-directed mutagenesis can be used to introduce mutations that create new restriction sites, alter glycosylation patterns, change codon preference, produce splice variants, and so forth.

In order to express a biologically active protein, the polynucleotide or derivatives thereof, can be inserted into an expression vector which contains the elements for transcriptional and translational control of the inserted coding sequence in a particular host. These elements can include regulatory sequences, such as enhancers, constitutive and inducible promoters, and 5′ and 3′ untranslated regions. Methods which are well known to those skilled in the art can be used to construct such expression vectors. These methods include in vitro recombinant DNA techniques, synthetic techniques, and in vivo genetic recombination (Sambrook, supra; Ausubel, supra).

A variety of expression vector/host cell systems can be utilized to express the polynucleotide. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the polynucleotide can be transformed into cell lines using expression vectors which can contain viral origins of replication and/or endogenous expression elements and a selectable or visible marker gene on the same or on a separate vector. The invention is not to be limited by the vector or host cell employed.

In general, host cells that contain the polynucleotide and that express the protein can be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or amino acid sequences. Immunological methods for detecting and measuring the expression of the protein using either specific polyclonal or monoclonal antibodies are known in the art. Examples of such assays include 2D-PAGE, MS, ELISAs, RIAs, FACS, and arrays.

Host cells transformed with the polynucleotide can be cultured under conditions for the expression and recovery of the protein from cell culture. The protein produced by a transgenic cell can be secreted or retained intracellularly depending on the sequence and/or the vector used. As will be understood by those of skill in the art, expression vectors containing the polynucleotide can be designed to contain signal sequences which direct secretion of the protein through a prokaryotic or eukaryotic cell membrane.

In addition, a host cell strain can be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein can also be used to specify protein targeting, folding, and/or activity. Different host cells which have specific cellular machinery and characteristic mechanisms for post-translational activities (e.g., CHO, HeLa, MDCK, HEK293, and W138) are available from the ATCC (Manassas Va.) and can be chosen to ensure the correct modification and processing of the expressed protein.

In another embodiment of the invention, natural, modified, or recombinant nucleic acid sequences are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the polynucleotides, wholly or in part, are synthesized using chemical or enzymatic methods well known in the art (Caruthers et al. (1980) Nucleic Acids Symp Ser (7) 215-233; Ausubel, supra). For example, peptide synthesis can be performed using various solid-phase techniques (Roberge et al. (1995) Science 269:202-204), and machines such as the 431A peptide synthesizer (ABI) can be used to automate synthesis. If desired, the amino acid sequence can be altered during synthesis and/or combined with sequences from other proteins to produce a variant.

Screening, Diagnostics and Therapeutics

The polynucleotides are particularly useful as markers in diagnosis, prognosis, treatment, and selection and evaluation of therapies for breast cancer. The polynucleotides can also be used to screen a plurality of molecules for specific binding affinity. The assay can be used to screen a plurality of DNA molecules, mimetics, peptides, peptide nucleic acids, proteins, RNA molecules and transcription factors which regulate the activity of the polynucleotide in the biological system. An exemplary assay involves providing a plurality of molecules, comtacting the combination or a polynucleotide with the plurality of molecules under conditions to allow specific binding, and detecting specific binding to identify at least one molecule which specifically binds the polynucleotide.

Similarly proteins or peptides can be used to screen libraries of molecules or compounds in any of a variety of screening assays. The protein or peptide employed in such screening can be free in solution, affixed to an abiotic or biotic substrate (e.g. borne on a cell surface), or located intracellularly. Specific binding between the protein and the molecule can be measured. The assay can be used to screen a plurality of agonists, antagonists, antibodies, DNA molecules, peptides, peptide nucleic acids, proteins including transcription factors, enhancers, and repressors, RNA molecules, and small drug molecules or compounds, which specifically bind the protein. One method for high throughput screening using very small assay volumes and very small amounts of test compound is described in U.S. Pat. No. 5,876,946, incorporated herein by reference, which screens large numbers of molecules for enzyme inhibition or receptor binding.

In one preferred embodiment, the polynucleotides are used for diagnostic purposes to determine the absence, presence, or differential expression. Differential expression must be increased or decreased as compared to a standard that is selected from either control cells, normal tissue, or well characterized diseased tissue. The polynucleotide consists of complementary RNA and DNA molecules, branched nucleic acids, and/or peptide nucleic acids. In one alternative, the polynucleotides are used to detect and quantify gene expression in samples in which expression of the polynucleotide is indicative of breast cancer. In another alternative, the polynucleotide can be used to detect genetic polymorphisms associated with breast cancer. These polymorphisms can be detected in transcripts or genomic sequences.

The specificity of the probe is determined by whether it is made from a unique region, a regulatory region, or from a conserved motif. Both probe specificity and the stringency of hybridization or amplification (maximal, high, intermediate, or low) will determine whether the probe identifies only naturally occurring, exactly complementary sequences, allelic variants, or related sequences. Probes designed to detect related sequences should have at least 50% sequence identity and to detect a sequence having a polymorphism preferably 94% sequence identity.

Methods for producing hybridization probes include the cloning of the polynucleotide into vectors for the production of RNA probes. Such vectors are known in the art, are commercially available, and can be used to synthesize RNA probes in vitro by adding RNA polymerases and labeled nucleotides. Hybridization probes can incorporate nucleotides labeled by a variety of reporter groups including, but not limited to, radionuclides such as ³²P or ³⁵S, enzymatic labels such as alkaline phosphatase coupled to the probe via avidin/biotin coupling systems, fluorescent labels, and the like. The labeled polynucleotides can be used in Southern or northern analysis, dot or slot blot, or other membrane-based technologies; in PCR technologies; and in microarrays utilizing samples from subjects to detect differential expression.

The polynucleotide can be labeled by standard methods and added to a sample from a subject under conditions for the formation and detection of hybridization complexes. After incubation the sample is washed, and the signal associated with hybrid complex formation is quantitated and compared with a standard value. Standard values are derived from any control sample, typically one that is free of the suspect disease. If the amount of signal in the subject sample is altered in comparison to the standard value, then the presence of differential expression in the sample indicates the presence of the disease. Qualitative and quantitative methods for comparing the hybridization complexes formed in subject samples with previously established standards are well known in the art.

Such assays can also be used to evaluate the efficacy of a particular therapeutic treatment regimen in animal studies, in clinical trials, or to monitor the treatment of an individual subject. Once the presence of disease is established and a treatment protocol is initiated, hybridization or amplification assays can be repeated on a regular basis to determine if the level of expression in the subjects begins to approximate that which is observed in a healthy subject. The results obtained from successive assays can be used to show the efficacy of treatment over a period ranging from several days to many years.

The polynucleotides can be used as a group or alone for the diagnosis of breast cancer. The polynucleotides can also be used on a substrate such as microarray to monitor the expression patterns. The microarray can also be used to identify splice variants, mutations, and polymorphisms. Information derived from analyses of the expression patterns can be used to determine gene function, to understand the genetic basis of a disease, to diagnose a disease, and to develop and monitor the activities of therapeutic agents used to treat a disease. Microarrays can also be used to detect genetic diversity, single nucleotide polymorphisms which can characterize a particular population, at the genome level.

In yet another alternative, polynucleotides can be used to generate hybridization probes useful in mapping the naturally occurring genomic sequence. Fluorescent in situ hybridization (FISH) can be correlated with other physical chromosome mapping techniques and genetic map data as described in Heinz-Ulrich et al. (In: Meyers, supra, pp. 965-968).

In another embodiment, antibodies or Fabs comprising an antigen binding site that specifically binds the protein can be used for the diagnosis of diseases characterized by the over-or-under expression of the protein. A variety of protocols for measuring protein expression, including 2-D PAGE, MS, ELISAs, RIAs, FACS, and arrays are well known in the art and provide a basis for diagnosing differential, altered or abnormal levels of expression. Standard values for protein expression are established by combining samples taken from healthy subjects, preferably human, with antibody to the protein under conditions for complex formation. The amount of complex formation can be quantitated by various methods, preferably by photometric means. Quantities of the protein expressed in disease samples are compared with standard values. Deviation between standard and subject values establishes the parameters for diagnosing or monitoring disease. Alternatively, one can use competitive drug screening assays in which neutralizing antibodies capable of binding specifically with the protein compete with a test compound. Antibodies can be used to detect the presence of any peptide which shares one or more antigenic determinants with the protein. In one aspect, the antibodies of the present invention can be used for treatment or monitoring therapeutic treatment for breast cancer.

In another aspect, the polynucleotide, or its complement, can be used therapeutically for the purpose of expressing mRNA and protein, or conversely to block transcription or translation of the mRNA. Expression vectors can be constructed using elements from retroviruses, adenoviruses, herpes or vaccinia viruses, or bacterial plasmids, and the like. These vectors can be used for delivery of nucleotide sequences to a particular target organ, tissue, or cell population. Methods well known to those skilled in the art can be used to construct vectors to express nucleic acid sequences or their complements (see, e.g., Maulik et al. (1997) Molecular Biotechnology, Therapeutic Applications and Strategies, Wiley-Liss, New York N.Y.). Alternatively, the polynucleotide or its complement, can be used for somatic cell or stem cell gene therapy. Vectors can be introduced in vivo, in vitro, and ex vivo. For ex vivo therapy, vectors are introduced into stem cells taken from the subject, and the resulting transgenic cells are clonally propagated for autologous transplant back into that same subject. Delivery of the polynucleotide by transfection, liposome injections, or polycationic amino polymers can be achieved using methods which are well known in the art (See, e.g., Goldman et al. (1997) Nature Biotechnol 15:462-466). Additionally, endogenous gene expression can be inactivated using homologous recombination methods which insert an inactive gene sequence into the coding region or other targeted region of the polynucleotide (see, e.g. Thomas et al. (1987) Cell 51: 503-512).

Vectors containing the polynucleotide can be transformed into a cell or tissue to express a missing protein or to replace a nonfunctional protein. Similarly a vector constructed to express the complement of the polynucleotide can be transformed into a cell to down-regulate the protein expression. Complementary or antisense sequences can consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (see, e.g., Gee et al. In: Huber and Carr (1994) Molecular and Immunologic Approaches, Futura Publishing, Mt. Kisco N.Y., pp. 163-177).

Ribozymes, enzymatic RNA molecules, can also be used to catalyze the cleavage of mRNA and decrease the levels of particular mRNAs, such as those comprising the polynucleotides of the invention (see, e.g., Rossi (1994) Current Biology 4: 469-47). Ribozymes can cleave mRNA at specific cleavage sites. Alternatively, ribozymes can cleave mRNAs at locations dictated by flanking regions that form complementary base pairs with the target mRNA. The construction and production of ribozymes is well known in the art and is described in Meyers (supra).

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, can be included.

Further, an antagonist, or an antibody that binds specifically to the protein can be administered to a subject to treat breast cancer. The antagonist, antibody, or fragment can be used directly to inhibit the activity of the protein or indirectly to deliver a therapeutic agent to cells or tissues which express the protein. The therapeutic agent can be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein can be generated using methods that are well known in the art. Such antibodies can include, but are not limited to, polyclonal, monoclonal, chimeric, and single chain antibodies, Fab fragments, and fragments produced by a Fab expression library. Neutralizing antibodies, such as those which inhibit dimer formation, are especially preferred for therapeutic use. Monoclonal antibodies to the protein can be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used (see, e.g., Pound (1998) Immunochemical Protocols, Methods Mol Biol Vol. 80). Alternatively, techniques described for the production of single chain antibodies can be employed. Fabs which contain specific binding sites for the protein can also be generated. Various immunoassays can be used to identify antibodies having the desired specificity. Numerous protocols for competitive binding or immunoradiometric assays using either polyclonal or monoclonal antibodies with established specificities are well known in the art.

Yet further, an agonist of the protein can be administered to a subject to treat or prevent a disease associated with decreased expression, longevity or activity of the protein.

An additional aspect of the invention relates to the administration of a pharmaceutical or sterile composition, in conjunction with a pharmaceutically acceptable carrier, for any of the therapeutic applications discussed above. Such pharmaceutical compositions can consist of the protein or antibodies, mimetics, agonists, antagonists, or inhibitors of the protein. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a subject alone or in combination with other agents, drugs, or hormones.

The pharmaceutical compositions utilized in this invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions can contain pharmaceutically-acceptable carriers comprising excipients and auxiliaries which facilitate processing of the active compounds into preparations which can be used pharmaceutically. Further details on techniques for formulation and administration can be found in the latest edition of Remington's Pharmaceutical Sciences (Mack Publishing, Easton Pa.).

For any compound, the therapeutically effective dose can be estimated initially either in cell culture assays or in animal models such as mice, rats, rabbits, dogs, or pigs. An animal model can also be used to determine the concentration range and route of administration. Such information can then be used to determine useful doses and routes for administration in humans.

A therapeutically effective dose refers to that amount of active ingredient which ameliorates the symptoms or condition. Therapeutic efficacy and toxicity can be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above can be applied to any subject in need of such therapy, including, but not limited to, mammals such as dogs, cats, cows, horses, rabbits, monkeys, and most preferably, humans.

Stem Cells and Their Use

SEQ ID NOs: 1-4 can be useful in the differentiation of stem cells. Eukaryotic stem cells are able to differentiate into the multiple cell types of various tissues and organs and to play roles in embryogenesis and adult tissue regeneration (Gearhart (1998) Science 282:1061-1062; Watt and Hogan (2000) Science 287:1427-1430). Depending on their source and developmental stage, stem cells can be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells can be transformed with polynucleotides which can be transiently expressed or can be integrated within the cell as transgenes.

Embryonic stem (ES) cell lines are derived from the inner cell masses of human blastocysts and are pluripotent (Thomson et al. (1998) Science 282:1145-1147). They have normal karyotypes and express high levels of telomerase which prevents senescence and allows the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95:13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287:1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96:14482-14486). Neural stem cells, which produce neurons and glia, can also be induced to differentiate into heart, muscle, liver, intestine, and blood cells (Kuhn and Svendsen (1999) BioEssays 21:625-630); Clarke et al. (2000) Science 288:1660-1663; Gage (2000) Science 287:1433-1438; and Galli et al. (2000) Nature Neurosci 3:986-991).

Neural stem cells can be used to treat neurological disorders such as Alzheimer disease, Parkinson disease, and multiple sclerosis and to repair tissue damaged by strokes and spinal cord injuries. Hematopoietic stem cells can be used to restore immune function in immunodeficient subjects or to treat autoimmune disorders by replacing autoreactive immune cells with normal cells to treat diseases such as multiple sclerosis, scleroderma, rheumatoid arthritis, and systemic lupus erythematosus. Mesenchymal stem cells can be used to repair tendons or to regenerate cartilage to treat arthritis. Liver stem cells can be used to repair liver damage. Pancreatic stem cells can be used to replace islet cells to treat diabetes. Muscle stem cells can be used to regenerate muscle to treat muscular dystrophies. (See, e.g., Fontes and Thomson (1999) BMJ 319:1-3; Weissman (2000) Science 287:1442-1446; Marshall (2000) Science 287:1419-1421; Marmont (2000) Ann Rev Med 51:115-134.)

### EXAMPLES

It is to be understood that this invention is not limited to the particular devices, machines, materials and methods described. Although particular embodiments known at the time the invention was made are described, equivalent embodiments can be used to practice the invention. The described embodiments are provided to illustrate the invention and are not intended to limit the scope of the invention which is limited only by the appended claims.

I cDNA Library Construction

RNA was purchased from Clontech or isolated from breast tissues, some of which are described for their sequence expression in Example VI below. Some tissues were homogenized and lysed in guanidinium isothiocyanate; others were homogenized and lysed in phenol or a suitable mixture of denaturants, such as TRIZOL reagent (Invitrogen). The resulting lysates were centrifuged over CsCl cushions or extracted with chloroform. RNA was precipitated from the lysates with either isopropanol or sodium acetate and ethanol, or by other routine methods. Phenol extraction and precipitation of RNA were repeated as necessary to increase RNA purity.

In some cases, RNA was treated with DNAse. For most libraries, poly(A+) RNA was isolated using oligo d(T)-coupled paramagnetic particles (Promega, Madison Wis.), OLIGOTEX latex particles (Qiagen, Valencia Calif.), or an OLIGOTEX mRNA purification kit (Qiagen). Alternatively, RNA was isolated directly from tissue lysates using RNA isolation kits such as the POLY(A)PURE mRNA purification kit; Ambion, Austin Tex.).

In some cases, Stratagene (La Jolla Calif.) was provided with RNA and constructed the cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art. (See, e.g., Ausubel, 1997, supra, units 5.1-6.6). Reverse transcription was initiated using oligo d(T) or random primers. Synthetic oligonucleotide adapters were ligated to double stranded cDNA, and the cDNA was digested with the appropriate restriction enzyme(s). For most libraries, the cDNA was size-selected (300-1000 bp) using SEPHACRYL S1000, SEPHAROSE CL2B, or SEPHAROSE CL4B column chromatography (Amersham Biosciences (APB), Piscataway N.J.) or preparative agarose gel electrophoresis. cDNAs were ligated into compatible restriction enzyme sites of the polylinker of pBLUESCRIPT plasmid (Stratagene), pSPORT1 plasmid (Invitrogen), or pINCY (Incyte Genomics). Recombinant plasmids were transformed into competent E. coli cells including XL1-BLUE, XL1-BLUEMRF, or SOLR (Stratagene) or DH5α, DH10B, or ElectroMAX DH10B (Invitrogen).

II Isolation, Sequencing and Analysis of cDNA Clones,

Plasmids were recovered from host cells by either in vivo excision using the UNIZAP vector system (Stratagene) or cell lysis. Plasmids were purified using one of the following kits or systems: a Magic or WIZARD Minipreps DNA purification system (Promega); an AGTC Miniprep purification kit (Edge Biosystems, Gaithersburg Md.); and QIAWELL 8 plasmid, QIAWELL 8 Plus plasmid, QIAWELL 8 Ultra Plasmid purification systems or the REAL Prep 96 plasmid kit (Qiagen). Following precipitation, plasmids were resuspended in 0.1 ml of distilled water and stored, with or without lyophilization, at 4C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a Fluoroskan II fluorescence scanner (Labsystems Oy, Helsinki, Finland).

The cDNAs were prepared for sequencing using the CATALYST 800 preparation system (ABI) or the HYDRA microdispenser (Robbins Scientific) or MICROLAB 2200 system (Hamilton, Reno Nev.) systems in combination with the DNA ENGINE thermal cyclers (MJ Research, Watertown Mass.). The cDNAs were sequenced using the PRISM 373 or 377 sequencing systems (ABI) and standard ABI protocols, base calling software, and kits. In one alternative, cDNAs were sequenced using the MEGABACE 1000 DNA sequencing system (Molecular Dynamics). In another alternative, the cDNAs were amplified and sequenced using the PRISM BIGDYE Terminator cycle sequencing ready reaction kit (ABI). In yet another alternative, cDNAs were sequenced using solutions and dyes from APB.

In that the nucleic acid sequences presented in the Sequence Listing were prepared by automated methods, they may contain occasional sequencing errors and unidentified nucleotides (N) that reflect state-of-the-art technology at the time the polynucleotide was first sequenced. Occasional sequencing errors and Ns may be resolved and single nucleotide polymorphisms verified either by resequencing the cDNA or using algorithms to align and compare multiple cDNA or genomic sequences covering the region of interest.

The polynucleotide sequences derived from cDNA, extension, and shotgun sequencing were assembled and analyzed using a combination of software programs which utilize algorithms well known to those skilled in the art (Meyers, supra, pp 856-853).

III Assembly of Polynucleotides and Characterization of Sequences

The sequences used for co-expression analysis were assembled from EST sequences, 5′ and 3′ long read sequences, and full length coding sequences.

The polynucleotides of this application were compared with assembled consensus sequences or templates found in the LIFESEQ GOLD database (Incyte Genomics). Component sequences from polynucleotide, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP (Phil Green, University of Washington, Seattle WA). Bins with several overlapping component sequences were assembled using DEEP PHRAP (Green, supra). The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms (Incyte Genomics) that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FAST× against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. Nos. 08/812,290 and 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Miss.).

The BLAST software suite, freely available sequence comparison algorithms (NCBI, Bethesda Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The polynucleotide and any encoded protein were further queried against public databases such as the GenBank rodent, mammalian, vertebrate, prokaryote, and eukaryote databases, SwissProt, BLOCKS, PRINTS, PFAM, and Prosite.

IV Co-expression of Breast Cancer Diagnostic Markers

The co-expression patterns of the known breast cancer diagnostic marker genes with each other and with the polynucleotides of SEQ ID NO: 1-4 were produced using GBA. Table 3 shows the co-expression of the known breast cancer diagnostic marker genes and proteins with each other. The entries in the table indicate the probability (−log P) that the observed co-expression for each pair of genes is due to chance as measured by the Fisher Exact Test.

TABLE 3
Co-expression of known breast cancer diagnostic marker
genes (- log P).

| Gene name | Zn-α2 | hMAM |
|---|---|---|
| Zn-α2 | | |
| hMAM | 14 | |
| BPAG1 | 4.8 | 10 |

Table 4 shows the co-expression of the known breast cancer diagnostic marker genes and the polynucleotides, SEQ ID NOs: 1-4. The entries in the table indicate the probability (−log P) that the observed co-expression for each pair of genes is due to chance as measured by the Fisher Exact Test.

TABLE 4
Co-expression of known breast cancer diagnostic marker
genes and SEQ ID NOs: 1-4 (- log P).

| Polynucleotide | SEQ ID | Zn-α2 | hMAM | BPAG1 |
|---|---|---|---|---|
| 411152 | 3 | 6.7 | 6.2 | 3.5 |
| 238469 | 1 | 14 | 19 | 9.1 |
| 1135407 | 4 | 15 | 26 | 9.4 |
| 348845 | 2 | 8.6 | 9.5 | 6.2 |

V Descriptions of Known Breast Cancer Diagnostic Marker Genes

Table 5 below shows the descriptions and references for the known breast cancer diagnostic markers.

| Gene | Description and Reference |
|---|---|
| Zn-αa2 | Up-regulated by glucocorticoids and androgens in a specific set |
| | of human breast carcinomas (Lopez-Boado et al. (1994) Breast |
| | Cancer Res Treat 29:247-58) |
| hMAM | A superior marker of breast cancer cells in peripheral blood |
| | (Grunewald et al. (2000) Lab Invest 80:1071-7); mammoglobins |
| | 1 and 2 are specific and sensitive markers of micrometastases |
| | in breast cancer patients (Ooka et al. (2000) Oncol Rep |
| | 7:561-6) |
| BPAG1 | Not expressed in invasive breast cancer cells including |
| | carcinoma in situ, down regulation may be associated with loss |
| | of normal cytoarchitecture (Bergstraesser et al. (1995) Am J |
| | Pathol 147:1823-39) |

VI Expression of Polynucleotides in Breast Cancer

Using the data in the LIFESEQ GOLD database (Incyte Genomics), four polynucleotides that showed highly significant expression, a cutoff p-value of less than 0.00001 (P<1e⁻⁵), in breast cancer were identified. The statistical method presented in the DESCRIPTION OF THE INVENTION was used to identify these polynucleotides among approximately five million cDNAs assigned to one of the 40,285 gene bins. The method identified polynucleotides with highly specific expression in breast tissue and particularly in breast cancer tissues. Table 1 shows the expression for each polynucleotide as identified by its SEQ ID NO.

TABLE 1
POLYNUCLEOTIDES HIGHLY AND SPECIFICALLY EXPRESSED
IN BREAST AND BREAST CANCER TISSUES

| | (log 2) | | | # B | # B Libs | # B | | | | | |
|---|---|---|---|---|---|---|---|---|---|---|---|
| SEQ | B/θ | # B | # θ | Tumor | w/Other | Normal | | | | | |
| ID | (P) | Libs | Libs | Libs | Diseases | Libs | P B | P θ | A B | A θ | P value |
| 1 | 12.34 | 8 | 0 | 4 | 3 | 1 | 8 | 0 | 56 | 1158 | 3.7e−11 |
| 2 | 9.3 | 35 | 1 | 15 | 11 | 9 | 23 | 1 | 41 | 1157 | 1.1e−30 |
| 3 | 9.06 | 268 | 9 | 124 | 47 | 95 | 30 | 5 | 34 | 1157 | 4.2e−37 |
| 4 | 6.58 | 16 | 3 | 8 | 3 | 5 | 13 | 3 | 51 | 1155 | 3.3e−5 |

Legend:
Column 1 shows the SEQ ID NO; column 2, the expression ratio (log 2) of breast vs. non-breast, polynucleotide present; column 3, number of transcripts in breast libraries; column 4, number of transcripts in non-breast libraries; column 5, number of transcripts in breast tumor libraries, column 6, number of transcripts in diseased, non-breast libraries; column 7, number of transcripts in normal breast libraries; column 8, number of normal breast libraries, polynucleotide present;
column 9, number of non-breast libraries polynucleotide present; column 10, number of breast libraries, polynucleotide absent; column 11, number of non-breast libraries, polynucleotide absent; and column 12, P-value (Fisher-exact) breast vs. non-breast.

VII Transcript Imaging

The process of producing a comparative transcript image was described in U.S. Pat. No. 5,840,484, incorporated herein by reference. The general categories for which transcript image data are available include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract.

Table 2 shows the expression of SEQ ID NOs: 1-4 in breast tissue of the exocrine glands category of the LIFESEQ GOLD database (Incyte Genomics). The first column shows library name; the second column, the number of cDNAs sequenced in that library; the third column, the description of the library; the fourth column, absolute abundance of the transcript in the library; and the fifth column, percentage abundance of the transcript in the library.

TABLE 2
Transcript Images of Breast Specific Polynucleotide Expression

| | | | | |
|---|---|---|---|---|
| Library | cDNAs | Description of Tissue | Abund | % Abund |

SEQ ID NO:1 (Incyte ID 238469)

| | | | | |
|---|---|---|---|---|
| BRSTTUT18 | 3736 | tumor, ductal CA, 68F | 7 | 0.19 |
| BRSTTUT15 | 6535 | tumor, adenoCA, 46F, m/BRSTNOT17 | 5 | 0.08 |
| BRSTNOT24 | 4413 | NF breast disease, 46F | 3 | 0.07 |
| BRSTTMR01 | 1479 | mw/ductal adenoCA, 62F, RP | 1 | 0.07 |
| BRSTNOT16 | 4010 | papillomatosis, mw/lobular CA, 59F | 2 | 0.05 |
| BRSTNOT19 | 4019 | breast, mw/lobular CA, 67F | 2 | 0.05 |

SEQ ID NO:2 (Incyte ID 348845)

| | | | | |
|---|---|---|---|---|
| BRSTTUT18 | 3736 | tumor, ductal CA, 68F | 5 | 0.13 |
| BRSTTMR01 | 1479 | mw/ductal adenoCA, 62F, RP | 1 | 0.07 |
| BRSTTUT14 | 3949 | tumor, adenoCA, 62F, m/BRSTNOT14 | 2 | 0.05 |
| BRSTNOT24 | 4413 | NF breast disease, 46F | 2 | 0.04 |
| BRSTNOT14 | 3790 | mw/ductal adenoCA, CA in situ, 62F | 1 | 0.03 |
| BRSTTUT20 | 3868 | tumor, ductal adenoCA, 66F | 1 | 0.03 |

SEQ ID NO:3 (Incyte 411152)

| | | | | |
|---|---|---|---|---|
| BRSTTUT17 | 2690 | tumor, ductal CA, 65F | 1 | 0.04 |
| BRSTTUT18 | 3736 | tumor, ductal CA, 68F | 1 | 0.03 |
| BRSTNOT16 | 4010 | mw/lobular CA, 59F, m/BRSTTUT22 | 1 | 0.03 |
| BRSTNOT28 | 3734 | PF changes, 40F | 1 | 0.03 |
| BRSTTUT15 | 6535 | tumor, adenoCA, 46F, m/BRSTNOT17 | 1 | 0.02 |
| BRSTNOT27 | 3939 | mw/ductal CA, aw/node mets, 57F | 1 | 0.02 |
| BRSTTUT02 | 7066 | tumor, adenoCA, 54F, m/BRSTNOT03 | 1 | 0.01 |

SEQ ID NO:4 (Incyte 1135407)

| BRSTTUT14 | 3949 | tumor, adenoCA, 62F, m/BRSTNOT14 | 47 | 1.19 |
|---|---|---|---|---|
| BRSTTUT17 | 2690 | tumor, ductal CA, 65F | 20 | 0.74 |
| BRSTDIT01 | 3394 | PF changes, mw/intraductal cancer, 48F | 23 | 0.77 |
| BRSTTUT15 | 6535 | tumor, adenoCA, 46F, m/BRSTNOT17 | 38 | 0.60 |
| BRSTNOT05 | 13205 | mw/lobular CA, 58F, m/BRSTTUT03 | 42 | 0.31 |
| BRSTNOT01 | 4627 | 56F | 10 | 0.22 |
| BRSTNOT28 | 3734 | PF changes, 40F | 8 | 0.21 |
| *All mixed, pooled, normalized and subtracted libraries have been removed from the table. Diseases attributed to mixed or pooled samples cannot be considered specific as to source, and the relative expression patterns of the polynucleotide in such libraries cannot be considered specific. The expression data in normalized and subtracted libraries, that have had high copy number sequences removed before processing, are skewed so that there can be a higher representation of lower copy | | | | |
| # number sequences. | | | | |

As shown above, SEQ ID NOs: 1-3 had higher expression in ductal carcinoma and SEQ ID NO:4 was significantly expressed in adenocarcinoma and not expressed in the cytologically normal matched tissue, BRSTNOT14. SEQ ID NOs: 1-4 were not expressed in normal breast libraries, BRSTNOT25 and BRSTNOT35, made from tissues removed during breast reduction surgeries.

VIII Library Descriptions Relevant to Expression Analysis

Descriptions of breast cDNA libraries found in the transcript image above are presented to demonstrate the data shown in Example IV which was produced using THE METHOD described in the DESCRIPTION OF THE INVENTION. Descriptions are presented only once below.

SEQ ID NOs: 1, 2 and 3 (BRSTTUT18)

The BRSTIUT18 cDNA library was constructed using 1.0 μg of polyA RNA isolated from right breast tumor tissue removed from a 68-year-old female during modified radical mastectomy. Pathology indicated infiltrating, high grade, ductal carcinoma of the breast. The skin surface had a bruised appearance and on palpation, there was a firm nodule adjacent to the skin, 3.5 cm superior to the nipple. The breast parenchyma revealed a firm tumor mass surrounded by an abundant amount of thick fibrous breast tissue. The remaining breast parenchyma revealed areas of sclerosis. The nipple and dermis were free of tumor. The nodule, situated in the deep subcutaneous tissue, was formed by high grade tumor cells present in a solid sheet and cords that infiltrated into the adjacent fatty and fibroconnective tissue in an irregular and aggressive pattern. Sections of tumor included masses of tumor tissue in which there was a dense fibrocollagenous mass that was infiltrated with streams and cords of cells similar to other tumor areas. Sections remote to the principal tumor represent fat and fibrous breast tissue and were free of tumor. Multiple lymph nodes were negative for tumor, but show marked, histiocytic proliferation with some phagocytosis of brown pigment resembling lipofuscin. Estrogen receptors were positive; progesterone receptors and mutated p53 assay, negative.

SEQ ID NO:3 (BRSTTUT17)

The BRSTTUT17 cDNA library was constructed using 2 μg of polyA RNA isolated from left breast tumor tissue removed from a 65-year-old Caucasian female during a unilateral radical mastectomy. Pathology indicated invasive and in-situ grade 3, nuclear grade 2 ductal carcinoma, forming a mass in the central portion of the breast. Most of the tumor was comedo carcinoma in situ. The skin, nipple, and fascia were uninvolved, but a single axillary lymph node was reactive. The progesterone receptor was positive, the estrogen receptor, negative by immunoperoxidase staining. Patient history included hyperlipidemia and uterine leiomyoma, and previous surgeries included breast biopsy, cholecystectomy, hysterectomy, bilateral salpingo-oophorectomy, and incidental appendectomy. The patient was taking tamoxifen. Family history included stomach cancer in the mother; myocardial infarction, atherosclerotic coronary artery disease, and prostate cancer in the father; and benign hypertension, breast cancer and hyperlipidemia in sibling(s).

SEQ ID NO:4 (BRSTTUT14 v BRSTNOT14)

The BRSTTUT14 cDNA library was constructed using 7.5 ng of polyA RNA isolated from breast tumor tissue removed from a 62-year-old Caucasian female during a unilateral extended simple mastectomy. Pathology indicated an invasive grade 3, nuclear grade 3 adenocarcinoma, ductal type, located in the upper outer quadrant. Ductal carcinoma in situ, comedo type, comprised 60% of the tumor mass. This tumor was localized far from a previous healing biopsy site, which showed no residual carcinoma. No angiolymphatic invasion was seen. The skin, nipple, and deep margins of resection were free of tumor. Metastatic adenocarcinoma was identified in one (of 14) axillary lymph nodes with no perinodal extension. Immunohistochemical stains showed the tumor cells were strongly positive for estrogen receptors and weakly positive for progesterone receptors. The patient presented with a lump in the breast and breast pain. Patient history included a benign colon neoplasm, hyperlipidemia, cardiac dysrhythmia, a normal delivery, alcohol abuse, and obesity. Patient medications included estrogen therapy, which had been discontinued. Family history included atherosclerotic coronary artery disease in the father; atherosclerotic coronary artery disease in the mother; myocardial infarction, colon cancer, ovary cancer, and lung cancer in the sibling(s); and a myocardial infarction and cerebrovascular disease in the grandparent(s).

The BRSTNOT14 cDNA library was constructed with microscopically normal breast tissue from the same donor.

IX Hybridization Technologies and Analyses

Immobilization of Polvnucleotides on a Substrate

The polynucleotides are applied to a substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris-HCl, pH 8.0), and twice in 2× SSC for 10 min each. The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0. 1% SDS and acetone, etching in 4% hydrofluoric acid (VWR Scientific Products, West Chester Pa.), coating with 0.05% aminopropyl silane (Sigma-Aldrich) in 95% ethanol, and curing in a 110C oven. The slides are washed extensively with distilled water between and after treatments. The nucleic acids are arranged on the slide and then immobilized by exposing the array to UV irradiation using a STRATALINKER Uv-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C; then the arrays are washed in 0.2% SDS and rinsed in distilled water as before.

Probe Preparation for Membrane Hybridization

Hybridization probes derived from the polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100C for five min, and briefly centrifuging. The denatured polynucleotide is then added to a REDIPRIME tube (APB), gently mixed until blue color is evenly distributed, and briefly centrifuged. Five μl of [³²P]dCTP is added to the tube, and the contents are incubated at 37C for 10 min. The labeling reaction is stopped by adding 5 μl of 0.2M EDTA, and probe is purified from unincorporated nucleotides using a PROBEQUANT G-50 microcolumn (APB). The purified probe is heated to 100C for five min, snap cooled for two min on ice, and used in membrane-based hybridizations as described below.

Probe Preparation for Polymer Coated Slide Hybridization

Hybridization probes derived from mRNA isolated from samples are employed for screening polynucleotides of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTF, 3 μl Cy3 or Cy5 labeling mix, 1 μl RNAse inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85C, and probes are purified using two successive CHROMASPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C for 16 hr. Following hybridization, the membrane is washed for 15 min at 25C in 1 mM Tris (pH 8.0), 1% Sarkosyl, and four times for 15 min each at 25C in 1 mM Tris (pH 8.0). To detect hybridization complexes, XOMAT-AR film (Eastman Kodak, Rochester N.Y.) is exposed to the membrane overnight at −70C, developed, and examined visually.

Polymer Coated Slide-based Hybridization

Probe is heated to 65C for five min, centrifuged five min at 9400 rpm in a 5415C microcentrifuge (Eppendorf Scientific, Westbury N.Y.), and then 18 μl are aliquoted onto the array surface and covered with a coverslip. The arrays are transferred to a waterproof chamber having a cavity just slightly larger than a microscope slide. The chamber is kept at 100% humidity internally by the addition of 140 μl of 5× SSC in a corner of the chamber. The chamber containing the arrays is incubated for about 6.5 hr at 60C. The arrays are washed for 10 min at 45C in 1× SSC, 0.1% SDS, and three times for 10 min each at 45C in 0.1× SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of polynucleotides in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon W095/35505).

Hybridization complexes are detected with a microscope equipped with an INNOVA 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

X Complementary Molecules

Molecules complementary to the polynucleotide, from about 5 nucleotides to about 5000 nucleotides, are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the polynucleotide encoding the protein.

XI Protein Expression

The protein encoded by SEQ ID NO:3 (Open reading frame=A66 to A895) is characterized by a potential cAMP- and cGMP-dependent protein kinase phosphorylation site at S₂₄₃; potential casein kinase II phosphorylation sites at S₃₆, S₄₂, S₄₈, S₁₁₂, S₁₆₁, and S₁₆₇; potential protein kinase C phosphorylation sites at T₁₇, S₄₈, T₁₀₂, T₁₃₆, S₁₆₁, S₁₆₇, S₁₈₆ and T₂₂₀. It is expressed by transforming the pINCY vector into competent E. coli cells using protocols well known in the art (Ausubel, supra, unit 16, incorporated by reference).

Expression and purification of the protein are achieved using either a cell expression system or an insect cell expression system. The pUB6/V5-His vector system (Invitrogen) is used to express protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies

XII Production of Antibodies

The protein is purified using polyacrylamide gel electrophoresis and used to immunize mice or rabbits. Antibodies are produced using the protocols below. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using a 431A peptide synthesizer (ABI) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the polynucleotide or Protein

The polynucleotide or the protein are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled polynucleotide or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories, Palo Alto Calif.), is used to screen for peptides that bind the protein of the invention. A polynucleotide encoding the protein is inserted into the multiple cloning site of a pLexA vector, ligated, and transformed into E. coli. A cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30C until the colonies have grown up and are counted. The colonies are pooled in a minimal volume of 1× TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30C. A sample of the culture is plated on SD/-Trp/-Ura media and incubated at 30C until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a polynucleotide encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A combination comprising a plurality of polynucleotides wherein the polynucleotides have the nucleic acid sequences of SEQ ID NOs: 1-4 and the complete complements of SEQ ID NOs: 1-4.

2. A substrate upon which the combination of claim 1 is immobilized.

3. A method for detecting gene expression in a sample containing nucleic acids, the method comprising:
a) hybridizing the substrate of claim 2 to the nucleic acids under conditions for formation of one or more hybridization complexes; and
b) detecting hybridization complex formation, wherein complex formation indicates gene expression in the sample.

4. The method of claim 3 wherein the sample is from breast.

5. The method of claim 3 wherein gene expression is compared to a standard and is indicative of breast cancer.

6. The method of claim 3 wherein the nucleic acids of the sample are amplified before hybridization.

7. A method for screening a plurality of molecules to identify at least one ligand which specifically binds a polynucleotide of the combination, the method comprising:
a) combining the substrate of claim 2 with molecules under conditions to allow specific binding; and
b) detecting specific binding, thereby identifying a ligand which specifically binds a polynucleotide of the combination.

8. The method of claim 7 wherein the molecules are selected from DNA molecules, mimetics, peptides, peptide nucleic acids, proteins, RNA molecules, ribozymes, and transcription factors.

9. An isolated polynucleotide comprising a nucleic acid sequence selected from SEQ ID NOs: 1-4 and the complements thereof.

10. A composition comprising a polynucleotide of claim 9 and a labeling moiety.

11. A method for using a polynucleotide to detect gene expression in a sample containing nucleic acids, the method comprising:
a) hybridizing the composition of claim 10 to nucleic acids of the sample under conditions for formation of one or more hybridization complexes; and
b) detecting hybridization complex formation, wherein complex formation indicates gene expression in the sample.

12. The method of claim 11, wherein the polynucleotide is attached to a substrate.

13. The method of claim 11, wherein gene expression is compared to a standard and is indicative of breast cancer.

14. A method of using a polynucleotide to screen a plurality of molecules to identify and purify a molecule which specifically binds the polynucleotide, the method comprising:
a) combining the polynucleotide of claim 9 with a plurality of molecules under conditions to allow specific binding;
b) recovering the bound polynucleotide; and
c) separating the ligand from the bound polynucleotide, thereby obtaining a purified molecule which specifically binds the polynucleotide.

15. The method of claim 14 wherein the molecules are selected from DNA molecules, mimetics, peptides, peptide nucleic acids, proteins, RNA molecules, ribozymes, and transcription factors.

16. A vector comprising a polynucleotide of claim 9.

17. A host cell comprising the vector of claim 16.

18. A method for using a host cell to produce a protein, the method comprising:
a) culturing the host cell of claim 17 under conditions for expression of the protein; and
b) recovering the protein from cell culture.

19. A purified protein obtained using the method of claim 18.

20. A composition comprising the protein of claim 19 and a pharmaceutical carrier.

21. A method for using a protein to screen a plurality of molecules to identify at least one ligand which specifically binds the protein, the method comprising:
a) combining the protein of claim 19 with the plurality of molecules under conditions to allow specific binding; and
b) detecting specific binding, thereby identifying a ligand which specifically binds the protein.

22. The method of claim 21 wherein the plurality of molecules is selected from agonists, antagonists, antibodies, DNA molecules, peptides, peptide nucleic acids, proteins including transcription factors, enhancers, and repressors, RNA molecules, and small drug molecules or compounds.

23. A method of using a protein to prepare and purify antibodies comprising:
a) immunizing an animal with the protein of claim 19 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified antibodies.

24. An antibody which specifically binds a protein produced by the method of claim 23.
